(19) United States
(12) Patent Application Publication
(10) Pub. No.: US 2023/0370793 A1
(43) Pub. Date: Nov. 16, 2023

(54) ACTIVE IMPLANT WITH PERCUTANEOUS ABUTMENT

(71) Applicant: Cochlear Limited, Macquarie University, NSW (AU)

(72) Inventors: Marcus ANDERSSON, Goteborg (SE); Marcus VARDFJÄLL, Askim (SE); Kenneth OPLINGER, Macquarie University, NSW (AU)

(21) Appl. No.: 18/246,421

(22) PCT Filed: Sep. 2, 2021
(86) PCT No.: PCT/IB2021/058027

§ 371 Date: Mar. 23, 2023

Related U.S. Application Data
(60) Provisional application No. 63/086,180, filed on Oct. 1, 2020.

(51) Int. Cl. H04R 25/00

(52) U.S. Cl. CPC H04R 25/606; H04R 2460/13

(57) ABSTRACT

An example apparatus includes a bone fixture, an active implant, a percutaneous abutment, and an external device. The percutaneous abutment electrically connects the external device with the active implant, such as for the transmission of power or data. In some examples, the percutaneous abutment itself includes one or more microphones, antennas or other components that provide functionality to the active implant. Data obtained from one or both of the external device and the percutanous abutment can be used to cause the active implant to perform a function, such as actuating a bone conduction vibratory actuator.

### BACKGROUND

### Field of the Invention

The present invention relates generally active implants with percutaneous abutments.

### Related Art

Medical devices have provided a wide range of therapeutic benefits to recipients over recent decades. Medical devices can include internal or implantable components/devices, external or wearable components/devices, or combinations thereof (e.g., a device having an external device communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices”, now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In an example, there is an apparatus comprising a bone fixture configured to anchor to bone of a recipient, a percutaneous abutment configured to mechanically couple to the bone fixture, and an active implant configured to be anchored by the bone fixture or the percutaneous abutment at a location least partially between the bone fixture and the percutaneous abutment.

In another example, there is a system comprising: a percutaneous abutment comprising at least one supracutaneous electrical contact and at least one subcutaneous electrical contact; an active implant comprising an implant battery and an active implant electrical contact configured to electrically couple to the at least one subcutaneous electrical contact; and an external device comprising at least one external device electrical contact configured to electrically couple to the at least one supracutaneous electrical contact, wherein the external device is configured to charge the implant battery via the at least one supracutaneous electrical contact.

In a further example, there is an apparatus comprising: a bone fixture configured to anchor to bone of a recipient; a percutaneous abutment coupled to the bone fixture; and an active implant disposed coaxially with the bone fixture and the percutaneous abutment, wherein the active implant comprises a vibratory actuator.

### BRIEF DESCRIPTION OF THE DRAWINGS

The same number represents the same element or same type of element in all drawings.

FIG. 1 illustrates a partial cross section view of a first example apparatus having an external device, a percutaneous abutment, an active implant, and a bone fixture.

FIG. 2 illustrates a cross section view of a second example apparatus including a percutaneous abutment, an active implant, and a bone fixture coupled via a threaded fastener.

FIG. 3 illustrates a partial cross-sectional view of a third example apparatus having an external device, an active implant, a bone fixture, and a percutaneous abutment having two bends.

FIG. 4 illustrates a fourth example apparatus having an external device in the form of a behind-the-ear device, a percutaneous abutment, an active implant, and a bone fixture.

FIG. 5 illustrates an example fifth example apparatus including an active implant having a rounded rectangle shape, a percutaneous abutment, and electrical contacts thereof.

### DETAILED DESCRIPTION

Disclosed examples include apparatuses having an active implant and a percutaneous abutment. In an example, the apparatus is a bone conduction device having a subcutaneous vibratory actuator (e.g., as part of the active implant) and a percutaneous abutment to which one or more components are connectable to cooperate with the active implant, such as by providing power and data to the active implant. In some examples, the percutaneous abutment itself includes one or more microphones, antennas or other components that provide functionality to the active implant. In many examples, the active implant is coupled to or anchored by the percutaneous abutment. In some examples, the apparatus can be used with recipients that have never received a bone conduction device before. In other examples, the apparatus can be retrofitted on recipients of existing percutaneous bone conduction devices.

In some examples, the size and weight of external devices can be reduced compared to traditional percutaneous bone conduction devices. Traditional percutaneous bone conduction devices lack active implantable components and therefore include their active components external to the recipient. Compared to such traditional examples, techniques described herein can be applicable to reducing the size and weight of components supported by the percutaneous abutment, thereby enabling a reduction in size of the percutaneous abutment itself. Reduced percutaneous abutment size is related to improved recipient comfort and can make the external portion of the apparatus more discreet. In some examples, the apparatus can be configured such that components with relatively short longevity (e.g., due to wear or short upgrade cycles) are disposed in an external device and relatively longer-term components are implanted. For instance, the transducer can be implanted while a battery, microphone, and sound processor being external can be beneficial because batteries (including rechargeable) are often changed more often than other parts. Further microphones can become worn with time and need changing. Sound processors often wear out less frequency but may nonetheless often be updated as new technology and features are added. Transducers are changed infrequently since transducers typically do not wear out or are upgraded as often as the other parts. Further, by being implanted a transducer is more protected from damage.

In some examples, one or more components of the bone conduction apparatus are mechanically connected using a separate screw that couples the abutment to the bone screw. In another example, the screw (or a thread) is integral with the percutaneous abutment. In a further example, the percutaneous abutment screws into the active implant and the active implant screws into bone screw.

In an example, a sound processor is implanted with the actuator in the active implant. Such an approach further reduces external device size. Further, such an approach can result in reduced external device cost and complexity because an external device can include, for example, just a battery and microphones as primary components and avoids system complexities due to an implanted battery. A further approach includes the recipient having multiple inexpensive, fully charged backup external devices handy to swap when the battery of the current device needs recharging. This approach can even permit the external battery to be smaller than typical batteries of percutaneous systems (e.g., having a battery capacity sufficient to run the apparatus for less than 16 hours), thereby further reducing external component size and increasing discretion. In a further example, the battery is implanted with the actuator and sound processor, thereby permitting the apparatus to be used in an implanted-battery-only mode, thus eliminating the need for a continuously-connected external device for the device to operate. In such an arrangement, the active implant itself can include one or more subcutaneous microphones. In such examples, an external charging module is configured to couple to the percutaneous abutment. The external charging module can include a battery to recharge the implanted battery of the implanted component. In some examples, the external charging module includes one or more microphones because, in some embodiments, the charging module covers or otherwise interferes with sound reaching the microphones embedded in the percutaneous abutment.

In some examples, the percutaneous abutment is an active component. In an example, the percutaneous abutment includes one or more microphones, an antenna for wireless communication, contacts for charging (e.g., the battery can be recharged by a pillow charger additionally or alternatively, such as is described in U.S. patent application Ser. No. 16/758,216, which is hereby incorporated herein by reference) and data transmission (all connected electrically via the abutment to the implanted components), and a coupling mechanism to couple a device to re-charge the implanted battery. In addition or instead, the percutaneous abutment can include, for example, at one or more supracutaneous microphones (e.g., two microphones on opposite sides of the percutaneous abutment) and electrical contacts to facilitate transmission of power and/or data to active implanted components. The contacts can be used by an external charging module or other module such as a communications or radio module to transmit energy or data to the active implant. In some examples, the percutaneous abutment is configured to extend laterally along the recipient's skull. For instance, a bone fixture is disposed closer to the ear than is typical for percutaneous devices, but the percutaneous abutment is coupled to the bone fixture and is configured to laterally extend away from the anchor and have a supracutaneous attachment disposed away from the bone fixture. In such an example, the supracutaneous portion is spaced apart from the recipient's outer ear.

In some examples, the apparatus is configured to address feedback from the actuator to the microphones. In an example, the percutaneous abutment is not configured to transmit vibrations. The percutaneous abutment can include a vibration damping section or be constructed from a vibration damping material. In some embodiments, a spring is integrated in the percutaneous abutment, which decouples the microphones from the vibrations and thereby reduces feedback. In other embodiments, the microphones are hard-coupled to the abutment, such that signal processing can address feedback. An example technique for using signal processing to reduce feedback for hard coupling is described in US 2014/0288357, which is hereby incorporated herein by reference in its entirety for any and all purposes. In some implementations, the percutaneous abutment is configured to be sufficiently damping to vibrations to avoid substantially interfering with one or more microphones. In still other implementations, the abutment can include at least two different portions: a stiff portion that couples to the implant and penetrates the skin and a hard rubber portion that houses the microphones and external coupling elements. This latter embodiment can be used to implement, for example, rotatable microphones and embedded electronics that work with the active implant (transmit data thereto) to ensure that the microphones are properly oriented.

### First Example Apparatus

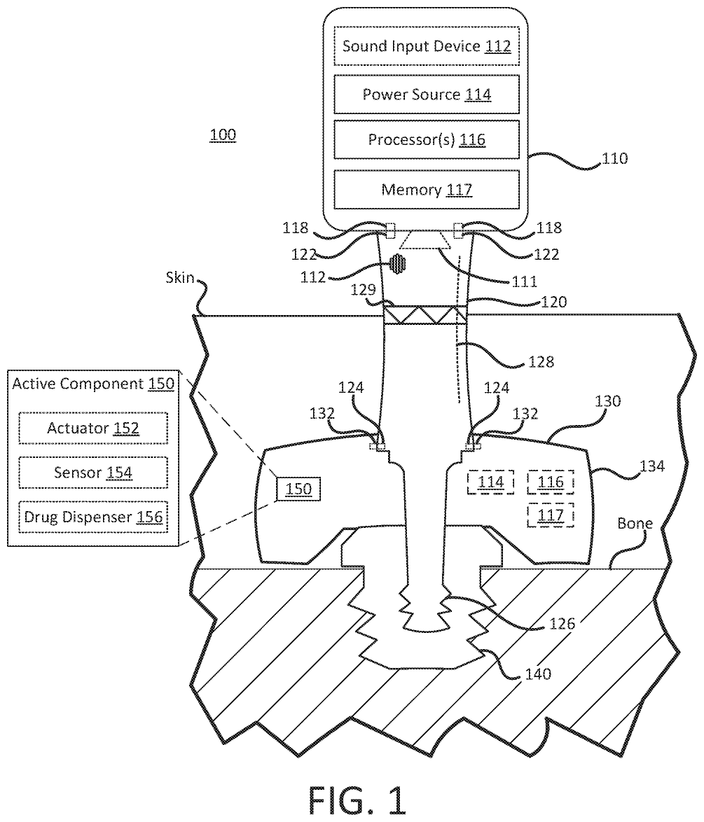

FIG. 1 illustrates a first apparatus 100 having an external device 110, a percutaneous abutment 120, an active implant 130, and a bone fixture 140. The apparatus 100 and its components are shown in partial cross section view.

### First Example Apparatus—External Device

The external device 110 is a component of the apparatus 100 configured to couple to the percutaneous abutment 120 and provide functionality. For example, the external device 110 establishes one or both of a mechanical connection and an electrical connection with the percutaneous abutment 120. For example, the external device 110 is configured to supply power and data to the active implant 130 via the electrical connection. In the illustrated example, the external device 110 includes one or more of a coupling 111, a sound input device 112, a power source 114, one or more processors 116, memory 117, and one or more external device electrical contacts 118, among other components.

The coupling 111 is a component of the external device 110 that is configured to couple with the percutaneous abutment 120. The coupling 111 can couple with the percutaneous abutment 120 in any of a variety of ways. In an example, the coupling 111 is a deformable component configured to form a snap-fit connection with the percutaneous abutment 120. In another example, the coupling 111 is a threaded connection configured to couple with the percutaneous abutment 120. In still further examples, the coupling 111 includes one or more magnets configured to establish a retentive magnet connection the percutaneous abutment 120. In some examples, the coupling 111 is a recessed area configured to receive a coupling of the percutaneous abutment 120. Other retention techniques or combinations thereof can be used.

A sound input device 112 is a component that receives sound into the apparatus, such as by converting acoustic energy into electric signals. The sound input device 112 can take any of a variety of forms, such as one or more microphones, auxiliary inputs, audio input ports, cable ports, telecoils, a wireless transceiver, accelerometers, other sound input devices, or combinations thereof. In some examples, the external device 110 includes multiple sound input devices 112. The multiple sound input devices 112 can be used to, for example, provide directionality, beamforming, noise cancelation, or other features.

The power source 114 is a component configured to provide operational power for one or more connected components. In an example, the power source 114 is in the form of one or more batteries or one or more capacitors. The power source 114 can be rechargeable or disposable. In some examples, the power source 114 is configured to store power for charging a power source of another device. For example, the power source 114 can be oversized with respect to the power requirements of the external device 110 so that the power source 114 is configured to charge a power source of the another device.

The one or more processors 116 are one or more electronic circuits that perform operations to control the performance of or be controlled by connected components (e.g., other components of the external device 110 or the apparatus 100 overall). For example, the one or more processors 116 can be or include one or more microprocessors (e.g., central processing units) or microcontrollers. In certain examples, the one or more processors 116 are implemented as one or more hardware or software processing units that obtain and execute instructions. The processors 116 can be configured to perform one or more methods or operations described herein. In an example, the one or more processors 116 are connected to the memory 117 having instructions encoded thereon that configure the processors 116 to perform a method. For instance, the memory 117 can include instructions that, when executed by the one or more processors 116 cause the one or more processors 116 to perform one or more operations described herein.

In an example, the one or more processors 116 are configured to implement a sound processor. For example, the sound processor 116 is configured to cause an actuator of the active implant 130 to actuate to cause a recipient of the apparatus 100 to experience a hearing percept. In addition or instead, the one or more processors 116 can be configured to obtain data from a sensor of the active implant 130 or to control a drug dispenser of the active implant 130.

The memory 117 can be one or more software- or hardware-based computer-readable storage media operable to store information. The memory 117 can be accessible by one or more of the processors 116. The memory 117 can store, among other things, instructions executable by the one or more processors 116 to cause performance of operations described herein. In addition or instead, the memory 117 can store other data. The memory 117 can be volatile memory (e.g., RAM), non-volatile memory (e.g., ROM), or combinations thereof. The memory 117 can include transitory memory or non-transitory memory. The memory 117 can include one or more removable or non-removable storage devices. The memory 117 can include RAM (Random Access Memory), ROM (Read Only Memory), EEPROM (Electronically-Erasable Programmable Read-Only Memory), flash memory, optical storage, magnetic storage, solid state storage, or any other memory media usable to store information for later access. In examples, the memory 117 encompasses a modulated data signal (e.g., a signal that has one or more of its characteristics set or changed in such a manner as to encode information in the signal), such as a carrier wave or other transport mechanism and includes any information delivery media. The memory 117 can include wired media such as a wired network or direct-wired connection, and wireless media such as acoustic, radiofrequency, infrared, other wireless media, or combinations thereof.

The one or more external device electrical contacts 118 are configured to electrically couple the external device 110 to another device or component. In examples, the one or more external device electrical contacts 118 of the external device 110 are configured to be compatible with one or more external contacts of another device, such as the percutaneous abutment 120. For example, the one or more external device electrical contacts 118 are configured to electrically couple to at least one supracutaneous electrical contact of the percutaneous abutment. For example, the one or more external device electrical contacts 118 are sized, shaped, and disposed on the external device 110 such that the one or more external device electrical contacts 118 align with and electrically connect to corresponding one or more supracutaneous electrical contacts when the external device 110 is coupled with the percutaneous abutment 120. The external device electrical contacts 118 can take any of a variety of forms, such as conductive pads, rings, plugs, other contacts, or combinations thereof. In some examples, one or more of the external device electrical contacts 118 are disposed on the coupling 111.

### First Example Apparatus—Percutaneous Abutment

The percutaneous abutment 120 is a percutaneous component of the apparatus 100 configured to directly or indirectly mechanically couple to the external device 110 and the bone fixture 140. The percutaneous abutment 120 is configured to be implanted with respect to a recipient such that a first portion of the percutaneous abutment 120 is subcutaneous and a second portion of the percutaneous abutment 120 is supracutaneous. In some implementations, the percutaneous abutment 120 acts as a bridge linking one or more external devices (e.g., the external device 110) with one or more implanted components (e.g., the active implant 130 and the bone fixture 140). In the illustrated example, the percutaneous abutment 120 has a substantially cylindrical shape and includes one or more supracutaneous sound input devices 112, one or more supracutaneous electrical contacts 122, one or more subcutaneous electrical contacts 124, a percutaneous abutment thread 126, one or more antennas 128, and one or more vibration dampers 129.

In an example, the one or more supracutaneous sound input devices 112 of the percutaneous abutment 120 are configured to supply data to one or both of the active implant 130 and the external device 110. The supracutaneous sound input devices 112 can include one or more features of the sound input devices 112 of the external device 110. In some examples, the one or more sound input devices 112 are one or more microphones disposed such that the microphones are covered or otherwise interfered with by a component being coupled to the supracutaneous portion of the percutaneous abutment 120. In some examples, the one or more microphones are disposed such that the vibration damper 129 is disposed between the one or more microphones and the active implant 130 when the apparatus 100 is implanted. The one or more supracutaneous sound input devices 112 can be electrically coupled to one or more of the supracutaneous electrical contacts 122 and the subcutaneous electrical contacts 124, such that one or more devices coupled thereto (e.g., the external device 110 and the active implant 130, respectively) can receive signals from the one or more supracutaneous sound input devices 112. While the sound input devices 112 of the percutaneous abutment 120 have been referred to as supracutaneous, the sound input devices 112 need not be supracutaneous. One or more of the sound input devices 112 can be subcutaneous or percutaneous.

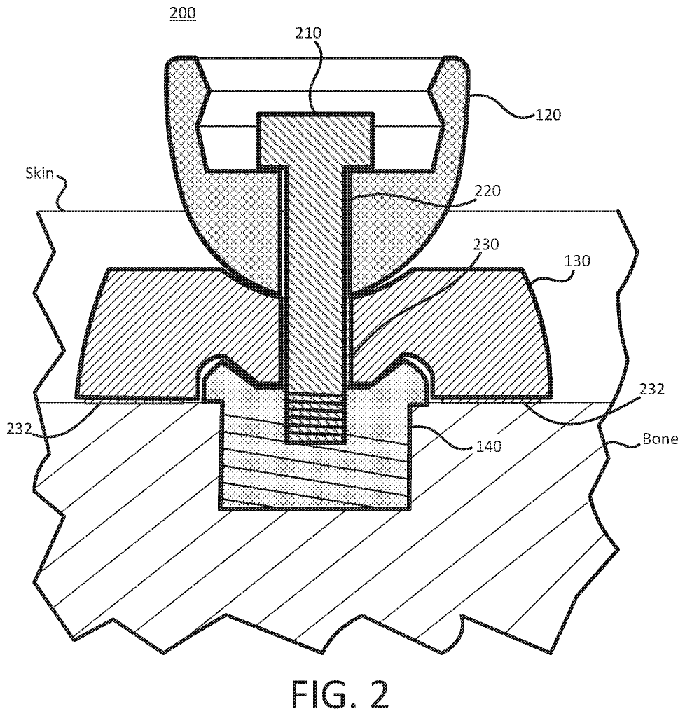

In an example, the percutaneous abutment thread 126 is configured to mate with a bone fixture thread to mechanically couple the percutaneous abutment 120 to the bone fixture 140. In this example, the percutaneous abutment 120 is mechanically coupled directly to the bone fixture 140. In other examples, the percutaneous abutment 120 can be mechanically coupled to the bone fixture 140 via another device, such as a fastener (see, e.g., fastener 210 of FIG. 2). In some examples, the percutaneous abutment thread 126 is configured to mate with a complimentary thread of the active implant 130 (which itself couples to the bone fixture 140, thereby indirectly securing the percutaneous abutment 120 to the bone fixture 140).

The illustrated percutaneous abutment 120 electrically couples the external device 110 to the active implant 130. The percutaneous abutment 120 comprises supracutaneous electrical contacts 122 and subcutaneous electrical contacts 124 configured to establish an electrical connection between an external device 110 and the active implant 130 when the external device 110 is coupled to the percutaneous abutment 120. In particular, the one or more supracutaneous electrical contacts 122 are configured to couple to the external device 110 and the one or more subcutaneous electrical contacts 124 are coupled to the active implant 130. In examples, the one or more electrical contacts 122, 124 of are configured to be compatible with one or more external contacts of another device. For example, the supracutaneous electrical contacts 122 can be configured to be compatible with the external device electrical contacts 118 of the external device 110. The subcutaneous electrical contacts 124 can be configured to be compatible with contacts of the active implant 130.

The one or more antennas 128 are one or more components or features of the percutaneous abutment 120 configured to receive signals. For example, the one or more antennas 128 can be configured to receive one or more radiofrequency signals (e.g., signals according to BLUETOOTH, WI-FI, AM, or FM protocols). The one or more antennas 128 can be electrically coupled to one or more of the supracutaneous electrical contacts 122 and the subcutaneous electrical contacts 124, such that one or more devices coupled thereto (e.g., the external device 110 or the active implant 130) can receive signals from the one or more supracutaneous sound input devices 112.

A vibration damper 129 is a component configured to resist transmitting vibrations, such as can be produced by the active implant 130. The vibration damper 129 can disposed in any of a variety of locations. In some examples, a vibration damper 129 is disposed to resist the transmission of vibrations across, through, or along the vibration damper 129, such as by resisting the transmission of vibrations from the active implant 130 to the sound input device 112 of the percutaneous abutment or the external device 110. The illustrated example includes a vibration damper 129 disposed between the active implant 130 and the sound input device 112 of the percutaneous abutment 120. The vibration damper 129 can be configured to resist the transmission of vibrations to or through the percutaneous abutment 120. The vibration damper 129 can be configured to vibrationally-decouple one or more parts of the apparatus 100.

The vibration damper 129 can take any of a variety of forms. For instance, the vibration damper 129 can be constructed to be an elastic, soft, flexible, non-rigid, gooey, and/or compliant component. In an example, the vibration damper 129 is formed as a suspension system that suspends the external device 110 away from the recipient's skin. The vibration damper 129 can include one or more springs. In some examples, the vibration damper 129 is disposed such that the vibration damper 129 extends at least partially through the recipient's skin when the apparatus 100 is implanted in the recipient. The vibration damper 129 being in contact with the recipient's skin after implantation can improve comfort of the percutaneous abutment 120 for the recipient.

The vibration damper 129 can be configured to deliberately damp, resist, inhibit, and/or attenuate vibration transfer through the vibration damper 129. The vibration damper 129 can be configured to do so through its construction from particular materials. In examples, the vibration damper 129 can be configured to attenuate the vibrations by at least 10%, at least 20%, at least 30%, at least 40%, at least 50%, at least 60%, or at least 70% (e.g., as measured via bench testing). For example, the attenuation can be measured by comparing an amount of vibrations transmitted through the vibration damper 129 compared to an original amount of vibrations before the effect of the vibration damper 129.

In some examples, the vibration damper 129 is a component of the percutaneous abutment 120. In addition or instead, the percutaneous abutment is constructed in such a manner as to act as the vibration damper 129. For example, the percutaneous abutment 120 can be constructed from a material being configured to resist transmitting vibrations. In some examples, the external device 110 is vibrationally isolated from the active implant 130 due to the vibration damper 129.

### First Example Apparatus—Active Implant

The active implant 130 is an implantable component configured to provide functionality, such as provide treatment to a recipient, deliver drugs to a recipient, or obtain medical data from a recipient, provide other functionality, or combinations thereof. The active implant 130 can be component that effects the purpose of the apparatus. In many examples herein, the active implant 130 is configured as an auditory prosthesis. In addition to or instead of an auditory prosthesis, the active implant 130 can take other forms, such as a drug-delivery device or a sensor. In the illustrated example, the active implant 130 has a cylindrical or ring shape and includes a power source 114, one or more processors 116, an active implant electrical contact 132, an active implant housing 134, and an active component 150.

The power source 114 of the active implant 130 is a component configured to provide operational power for one or more connected components of the active implant 130. In some examples, the active implant 130 lacks a power source 114 disposed within the active implant housing 134 and instead relies on one or more other power sources 114 for power. For example, the active implant 130 can be configured to receive operational power from the external device 110.

The one or more processors 116 can include one or more features of the one or more processors 116 of the external device 110. In some examples, the active implant 130 lacks any processor 116 and is controlled by the external device 110 or another component coupled with the active implant 130.

In an example, the active implant electrical contact 132 is configured to electrically couple to the at least one subcutaneous electrical contact 124 of the percutaneous abutment 120. In an example, the active implant electrical contact 132 is configured to be compatible with one or more electrical contacts of the percutaneous abutment 120.

The active implant housing 134 is a biocompatible housing of the active implant 130. The one or more components of the active implant 130 can be disposed in or coupled to the active implant housing 134.

The active implant 130 is the set of one or more components of the active implant that provide functionality, such as provide treatment to a recipient, deliver drugs to a recipient, or obtain medical data from a recipient, among others or combinations thereof. Example treatment include causing the recipient to experience sensory percepts, such as hearing percepts or vestibular percepts. In the illustrated example, the active component 150 includes one or more of an actuator 152, a sensor 154, and a drug dispenser 156.

The actuator 152 can be a bone conduction transducer configured to actuate to cause a recipient of the active implant 130 to experience a hearing percept. In an example, the actuator 152 is vibratory actuator, such as to implement a bone conduction auditory prosthesis. In an example, the actuator 152 is a piezoelectric actuator. The vibratory actuator 152 is configured to deliver vibrations directly to bone of the recipient or is configured to deliver vibrations to bone of the recipient via the bone fixture 140 or another component.

In the illustrated example, the active implant 130 is not directly in contact with bone of the recipient. Thus, the actuator 152 delivers vibrations to bone of the recipient indirectly through the bone fixture 140. In other examples, the active implant 130 includes a vibration-transfer surface configured to directly deliver vibrations to bone of the recipient.

The sensor 154 is a set of one or more components configured to generate signals based on an environment. Example sensors 154 include one or more: telecoils, biosensors (e.g., heart rate sensors or blood pressure sensors), glucose sensors (e.g., to provide a blood glucose signal), blood-alcohol sensors, one or more microphones (e.g., as described elsewhere herein), one or more other sensors, or combinations thereof.

The drug dispenser 156 is a component configured to deliver drugs to a recipient. In some examples, the drug dispenser 156 is configured as a drug pump. In some examples, the drug dispenser 156 is configured as a drug-eluding component.

As illustrated, the active implant 130 is a component that is discrete from the percutaneous abutment 120 and the bone fixture 140 and is entirely subcutaneous. In other examples, the active implant 130 can be integrated into one or both of the bone fixture 140 and the percutaneous abutment 120.

### First Example Apparatus—Bone Fixture

The bone fixture 140 is a component configured to anchor to a bone of a recipient. In many examples, the bone fixture 140 is an interface between the recipient's bone and one or both of the percutaneous abutment 120 and the active implant 130. The bone fixture 140 can take any of a variety of forms. An example implementation of a bone fixture is described in U.S. Pat. No. 9,838,807, which is hereby incorporated herein by reference in its entirety for any and all purposes. For instance, the bone fixture 140 is a screw-shaped anchoring fixture for anchoring the percutaneous abutment 120 in the recipient's skull bone. The bone fixture 140 has a main body configured to be implanted into the bone and a flange configured to function as a stop to prevent the main body from completely penetrating through the bone.

### First Example Apparatus—Physical Connections

The above-described components and one more other components can be configured to be combined to from the apparatus 100. In the example illustrated in FIG. 1, the external device 110 is configured to mechanically couple to the percutaneous abutment 120, the percutaneous abutment 120 is configured to mechanically couple to both the external device 110 and the bone fixture 140, and the active implant 130 is disposed between at least a portion of the percutaneous abutment 120 and the bone fixture 140. Other configurations are also possible.

In an example, the external device 110 can form one or both of a mechanical connection and an electrical connection with the percutaneous abutment 120. The mechanical connection between the external device 110 and the percutaneous abutment 120 is a connection that retains the external device 110 in a position relative to the percutaneous abutment 120. In some examples, the mechanical connection facilitates an electrical connection by placing electrical contacts in appropriate locations relative to each other. The mechanical connection can provide sufficient retention force to hold the external device 110 in a wearable relationship with the percutaneous abutment relative to the recipient's skull. For example, the mechanical connection between the external device 110 and the percutaneous abutment 120 can be sufficient to support the weight of the external device 110 during predetermined activities. The external device 110 can include one or more retention features to establish the mechanical connection, such as a coupling 111. As illustrated, the coupling 111 of the external device 110 can be configured as a snap fastener. In particular, the coupling 111 can couple with percutaneous abutment 120 by deforming when in substantial compressive contact with the percutaneous abutment 120, such as resulting from a user pressing the coupling 111 against the percutaneous abutment 120. Thus, when the coupling 111 is pressed into the percutaneous abutment 120, the coupling 111 elastically deforms radially inward to accommodate a lip or another attachment feature of the percutaneous abutment 120. Once a portion of the coupling 111 having the greatest diameter passes the lip, the coupling 111 begins to elastically expand radially outward and a radial retention surface of the coupling 111 presses against a surface of the percutaneous abutment 120 (e.g., a lip of a sidewall of the percutaneous abutment 120) so that the coupling 111 snaps into place, which still at least slightly limits the expansion of the coupling 111. In this manner, the coupling 111 presses against the percutaneous abutment 120 and retains the external device 110 relative to the percutaneous abutment 120, thereby establishing a mechanical connection between the external device 110 and the percutaneous abutment 120. Once coupled, the interaction between the coupling 111 and the percutaneous abutment 120 resists movement of the external device 110 relative to the percutaneous abutment 120. In particular, the coupling 111 resists movement unless a sufficient amount of force (e.g., removal force) is applied to cause the coupling 111 to deform to allow the coupling 111 to pass the lip. The mechanical connection provided by the coupling 111 can not only resist movement of the external device 110 relative to the percutaneous abutment 120, but it can also align electrical contacts to facilitate the formation of an electrical connection. In other examples, there can be a threaded connection or a magnetic connection between the external device 110 and the percutaneous abutment 120. Other kinds of connections are also possible.

In an example, the percutaneous abutment 120 is configured to be anchored by the bone fixture 140 via a direct or indirect connection with the bone fixture 140. As illustrated, the percutaneous abutment 120 is be coupled directly to the bone fixture 140 via connection between the percutaneous abutment thread 126 and a complimentary thread of the bone fixture 140. Other mechanical connections between the percutaneous abutment 120 and the bone fixture 140 are also possible. In some examples, the percutaneous abutment 120 is coupled indirectly to the bone fixture 140 via another component, such as a discrete threaded fastener as described in more detail in relation to FIG. 2. In some examples, the percutaneous abutment 120 is coupled to the bone fixture 140 via the active implant 130.

In the illustrated example, the active implant 130 defines a shaft through which an unthread portion of the percutaneous abutment 120 extends. The percutaneous abutment 120 can be threaded into the bone fixture 140, which clamps the active implant 130 between the percutaneous abutment 120 and the bone fixture 140. This clamping force can resist movement of the active implant 130 and place the active implant 130 in contact with the bone of the recipient or the bone fixture 140 itself in a way that provides sufficient vibratory transmission. An unthreaded shank portion of the percutaneous abutment 120 (or another component, such as a fastener) can be disposed within a shaft of the active implant 130 when the apparatus 100 is assembled and implanted. In an example, the active implant 130 is coupled directly to the percutaneous abutment 120 via a threaded connection.

In some examples, the mechanical connection between the percutaneous abutment 120, the active implant 130, and the bone fixture 140 creates a seal that resists entry of bodily fluids into a space between the components. For example, the seal can resist the entry of material that would substantially interfere with the electrical connection between the subcutaneous electrical contacts 124 and the active implant electrical contacts 132.

In the illustrated example, the active implant 130 is configured to be anchored by the bone fixture 140 or the percutaneous abutment 120 at a location least partially between the bone fixture 140 and the percutaneous abutment 120. For example, in the illustrated example, when viewed in a direction parallel with the recipient's bone into which the apparatus 100 is anchored and with the bone at the bottom of the view, the bone anchor 140 extends at least partially below the active implant 130 and the percutaneous abutment 120 extends at least partially above the active implant. The active implant 130 overlaps both the percutaneous abutment 120 and the active implant 130. In the illustrated example, the active implant 130, the bone fixture 140, and the percutaneous abutment 120 are disposed in a coaxial relationship. In other examples, the active implant 130 is not coaxial with one or both of the percutaneous abutment 120 and the active implant 130.

In an implementation, the percutaneous abutment 120 has a smaller diameter than the active implant 130 and the bone fixture 140.

In some examples, the active implant 130 is anchored directly to the recipient's skull. In an example, the active implant 130 is anchored by the bone fixture 140. In an example, the active implant 130 is anchored by the percutaneous abutment 120. Although specific examples have been provided above, the components can have any of a variety of relationships with respect to each other.

The mechanical connections between two or more of the components of the apparatus 100 can contribute to the forming of electrical connections between the two or more of the components.

### First Example Apparatus—Electrical Connections

As described above, various components of the apparatus 100 can include electrical contacts to establish electrical connections among the various components of the apparatus 100. Such connections can facilitate various functionality of the apparatus 100. In an example, the external device 110 is electrically coupled to the active implant 130 via the percutaneous abutment 120. In an example, the external device 110 is configured to supply power and data to the active implant 130 by being directly electrically coupled to the at least one supracutaneous electrical contact 122 of the percutaneous abutment 120. The at least one supracutaneous electrical contact 122 is electrically connected to at least one corresponding subcutaneous electrical contact 124 that is directly electrically coupled to at least one active implant electrical contact 132 of the active implant 130. In an example, the external device 110 is configured to electrically couple to the active implant 130 via the percutaneous abutment 120 to charge an implanted power source 114 of the active implant 130 from the external device power source 114. In an example, the external device 110 is configured to charge the implant power source 114 of the active implant 130 via the at least one supracutaneous electrical contact 122. For example, the power source 114 of the external device 110 is electrically connected to the percutaneous abutment 120 via a direct electrical connection between the one or more external device electrical contacts 118 and the one or more supracutaneous electrical contacts 122 of the percutaneous abutment 120. There can then be an electrical connection between the one or more supracutaneous electrical contacts 122 of the percutaneous abutment 120 and the one or more subcutaneous electrical contacts 124 of the percutaneous abutment 120. The one or more subcutaneous electrical contacts 124 can then be directly electrically connected to the one or more active implant electrical contacts 132, thereby establishing an electrical connection from the power source 114 of the external device 110 to the implant power source 114. A same or similar connection can be used to electrically connect one or more components of the external device 110 with the active implant 130, such as the sound input device 112 or one or more processors 116. Such a connection can be used to supply data in addition to or instead of supplying power.

In an example, one or both of the sound input device 112 and the antenna 128 of the percutaneous abutment 120 is electrically connected to the external device 110 via one or more supracutaneous electrical contacts 122. In an example, one or both of the sound input device 112 and the antenna 128 of the percutaneous abutment 120 are electrically connected to the active implant 130 via one or more supracutaneous electrical contacts 122.

### Apparatus—Example Use

The components of the apparatus 100 can cooperate to perform any of variety of operations.

In an example, the active implant 130 operates substantially independently. For example, the active implant powers itself for a time and perform one or more actions with the active component 150 without requiring input from another component of the apparatus. For example, where the active component 150 is a bone conduction device, the active implant 130 obtains audio data (e.g., with a sound input device 112 of the active implant 130), process the audio data with the one or more processors 116 of the active implant 130, and cause the recipient to experience an auditory percept by activating an actuator 152 of the active component 150 based on the processed audio data. When an external charging device 110 is coupled to the percutaneous abutment 120, the active implant 130 receives charging power from the external charging device 110 to charge the power source 114 of the active implant 130. The power source 114 of the active implant 130 can provide operational power to one or more other components of the active implant 130 such that the active implant 130 can operate without an external device 110 being connected for a period of time (e.g., at least four hours, at least eight hours, at least twelve hours, or at least sixteen hours).

In an example, the active implant 130 operates based on data received from one or more other components. For example, the apparatus 100 receives audio or other data (raw or processed) from the percutaneous abutment 120 or external device 110 and performs one or more operations based thereon, such as activating the active component 150. For instance, the apparatus 100 receive data using the sound input device 112 or antenna 128 of the percutaneous abutment 120 and transmit the received data to the active implant 130 via a direct or indirect electrical connection, and then the active implant 130 takes an action based thereon. In another example, the apparatus 100 receives data using the sound input device 112 of the external device 110 and transmits the received data to the active implant 130 via the percutaneous abutment 120, and then the active implant 130 takes an action based thereon.

### Second Example Apparatus

FIG. 2 illustrates a second example apparatus including a percutaneous abutment 120, an active implant 130, and a bone fixture 140 coupled via a threaded fastener 210. In an example, the threaded fastener 210 is a screw or bolt coupling the percutaneous abutment 120 to a bone fixture 140. The illustrated threaded fastener 210 extends through a percutaneous abutment shaft 220 of the percutaneous abutment 120 and an active implant shaft 230 of the active implant 130 to reach the bone fixture 140. The threaded fastener 210 is mated with a complimentary thread of the bone fixture 140, thereby the threaded fastener 210 couples the percutaneous abutment 120 to the bone fixture 140. In the illustrated example, the active implant 130, the percutaneous abutment 120, the threaded fastener 210, and the bone fixture 140 are coaxial. The connection between the threaded fastener 210 and the bone fixture 140 can form a clamping force that pulls the percutaneous abutment 120, active implant 130, and the bone fixture 140 together. The illustrated active implant 130 illustrates a tissue-contact surface 232 of the active implant 130. The tissue-contact surface 232 can be a surface of the active implant housing 134 or a component extending from the active implant 130 configured to be in contact with the recipient's skull or other tissue. The tissue-contact surface 232 can be mechanically coupled to the actuator 152 and be configured to transmit vibration output from the actuator 152 to the skull of the recipient.

### Third Example Apparatus

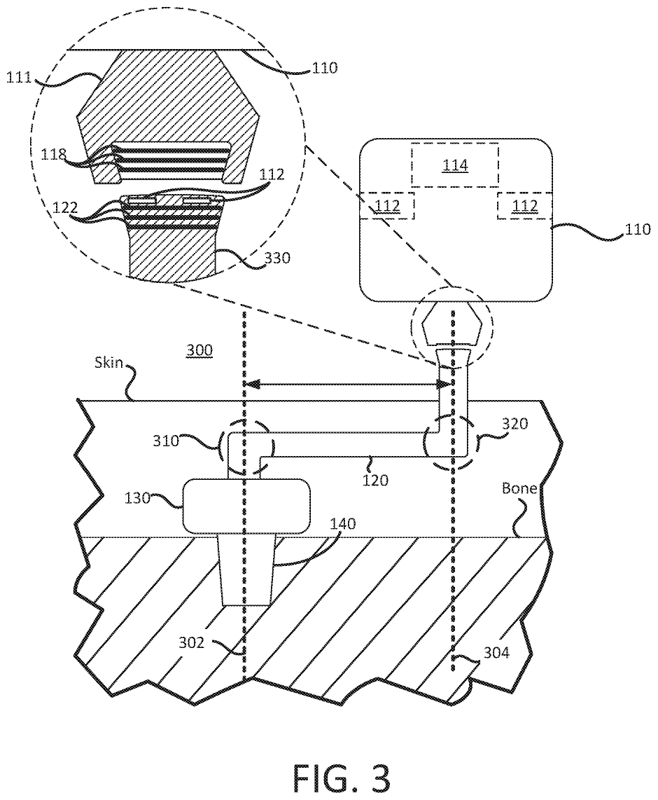

FIG. 3 illustrates a third example apparatus 300. As illustrated, the apparatus 300 defines is a first axis 302 and a second axis 304. The first axis 302 extends through (e.g., coaxially) one or both of the active implant 130 and the bone fixture 140. The second axis 304 extends through (e.g., coaxially) the external device 110 or a connection between the external device 110 and the percutaneous abutment 120. The active implant 130 and the external device 110 are configured to be laterally offset from each other along a recipient's skull when the active implant 130 is implanted in the recipient and the external device 110 is coupled to the percutaneous abutment 120. In the illustrated example, the components are configured to be laterally offset by the percutaneous abutment 120 extending laterally, such that the first axis 302 and the second axis 304 are separate and substantially parallel. In other examples, the first axis 302 and the second axis 304 are intersecting.

In the illustrated example, the percutaneous abutment 120 has a first bend 310 and a second bend 320. The bends 310, 320 are configured to the offset the active implant 130 and the external device 110 from each other along a recipient's skull when the active implant 130 is implanted in the recipient and the external device 110 is coupled to the percutaneous abutment 120. In an example, the bends 310, 320 are configured to laterally offset the first axis 302 from the second axis 304. In an example, the bends 310, 320 are configured to laterally offset the external device 110 from the active implant 130 with respect to a surface of the recipient's tissue (e.g., bone and skin). In an example, the bends 310, 320 are configured to laterally offset at least one supracutaneous electrical contact 122 and the at least one subcutaneous electrical contact 124 from each other.

A connection between the external device 110 and the percutaneous abutment 120 is also shown. The coupling 111 of the external device 110 is configured to receive a portion of a percutaneous abutment coupling 330. The interior of the coupling 111 includes multiple ring external device electrical contacts 118 configured to contact multiple ring electrical supracutaneous electrical contacts 122 of the percutaneous abutment 120. As further illustrated, the percutaneous abutment coupling 330 includes two microphones sound input devices 112 configured as microphones. The two sound input devices 112 of the percutaneous abutment coupling 330 are obstructed by the coupling 111 of the external device 110 when the coupling 111 and percutaneous abutment coupling 330 are coupled.

### Fourth Example Apparatus

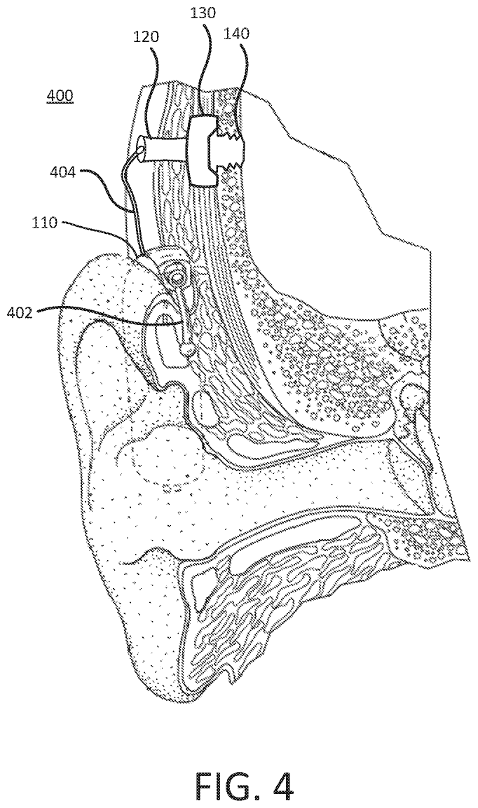

FIG. 4 illustrates a fourth example apparatus 400 that includes an external device 110 in the form of a behind-the-ear device, a percutaneous abutment 120, an active implant 130, and a bone fixture 140. The external device 110 is configured to be worn behind the recipient's ear, such as by being supported by an ear-hook 402 without being supported by the percutaneous abutment 120. The apparatus 400 includes a cable 404 electrically coupling the external device 110 and the percutaneous abutment 120. In some examples, the cable 404 is a component of the external device 110. In other examples, the cable 404 is a discrete component. The cable 404 can be configured to transmit or receive one or both of charging power and data. In addition, the cable 404 can be configured to resist transmission of vibrations from the percutaneous abutment 120 to the external device 110.

### Fifth Example Apparatus

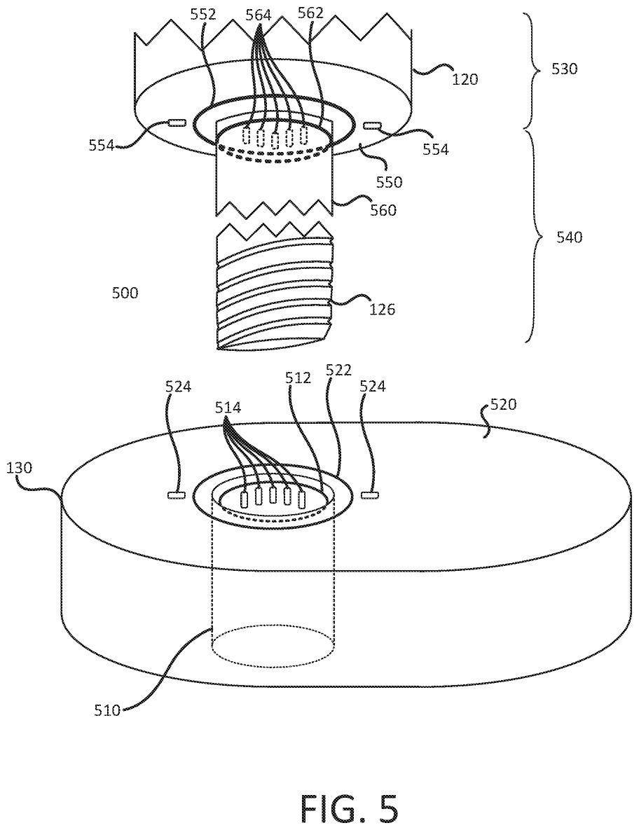

FIG. 5 illustrates an example fifth apparatus 500 including an active implant 130 having a rounded rectangle shape, a percutaneous abutment 120, and electrical contacts thereof.

As illustrated, sections of the percutaneous abutment 120 are illustrated. The illustrated percutaneous abutment 120 includes two different sections defined by differences in diameter. The first section 530 is a section having a greater diameter than the second section 540. In the illustrated example, the transition between the first section 530 and the second section 540 defines a transition surface 550. The illustrated percutaneous abutment 120 includes a transition ring electrical contact 552 and two transition electrical contact pads 554. As illustrated, the transition ring electrical contact 552 forms a partially or fully circular shape coaxial with the first section 530 and the second section 540. The transition ring electrical contact 552 has a diameter greater than the diameter of the second section 540 and less than a diameter of the first section 530. Although the illustrated example shows one transition ring electrical contact 552, other examples can include zero or one or more transition ring electrical contacts 552 and zero or one or more transition electrical contact pads 554.

In the illustrated example, the second section 540 includes a shank 560. Disposed on the shank portion are a shank ring electrical contact 562 and multiple shank electrical contact pads 564 disposed on a circumferential surface of the shank 560. Although the illustrated example shows one shank ring electrical contact 562 and multiple shank electrical contact pads 564, other examples can include zero or one or more shank ring electrical contact 562 and zero or one or more shank electrical contact pads 564.

As illustrated, the active implant 130 defines a shaft 510 that extends through the active implant 130 and a top surface 520. As illustrated, the apparatus 500 has various features useful for establishing electrical connections with the percutaneous abutment 120.

The shaft 510 is an opening extending through the active implant 130. The shaft 510 can be configured to receive the percutaneous abutment 120. As illustrated, the shaft 510 has an elongate, cylindrical shape. In other examples, the shaft 510 can include various protrusions, recesses, or other features (e.g., for interfacing with the percutaneous abutment or the bone fixture 140. As illustrated, there is at least one shaft ring electrical contact 512 of the active implant 130 disposed within the shaft 510. The at least one shaft ring electrical contact 512 is a conductive portion disposed within the shaft 510 of the active implant 130. For example, the at least one shaft ring electrical contact 512 is disposed circumferentially around at least a portion of the active implant 130 defining the shaft 510. Each of the at least one shaft ring electrical contacts 512 can be separated by an insulating section. Depending on the implementation, a shaft ring electrical contact 512 can benefit from not requiring a particular orientation of the percutaneous abutment 120 fitting through the shaft. As further illustrated, there are multiple, discrete shaft electrical contact pads 514 disposed within the portion of the active implant 130 defining the shaft 510. The shaft electrical contact pads 514 can be placed in electrical contact with a component disposed within the shaft 510, such as the percutaneous abutment 120.

The top surface 520 is a surface of the active implant 130 that faces toward the recipient's skin and away from the recipient's bone when implanted. In an example, the top surface 520 is the portion of the active implant 130 that faces the external device 110. As illustrated, the top surface 520 includes a top surface ring electrical contact 522 and a two top surface electrical contact pads 524, though other implementations can include more or fewer of the contacts 522, 524.

The percutaneous abutment 120 and the active implant 130 are illustrated as separate components. To connect the percutaneous abutment and the active implant, 130, the second section 540 of the percutaneous abutment 120 is inserted into the shaft 510 defined by the active implant 130. The transition surface 554 and the top surface 520 of the active implant 130 are then brought together. The various electrical contacts of the percutaneous abutment 120 and the active implant 130 can be configured such that when the percutaneous abutment 120 and the active implant 130 are brought together, associated electrical contacts are brought together in a useful manner to from an electrical connection therebetween. In some examples, the percutaneous abutment 120 and active implant 130 cooperate (e.g., via alignment features) so that the contacts line up appropriately.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

What is claimed is:

1. An apparatus comprising:
a bone fixture configured to anchor to bone of a recipient;
a percutaneous abutment configured to mechanically couple to the bone fixture; and
an active implant configured to be anchored by at least one of the bone fixture or the percutaneous abutment at a location least partially between the bone fixture and the percutaneous abutment.

2. The apparatus of claim 1, further comprising:
a threaded fastener coupling the percutaneous abutment to the bone fixture,
wherein the active implant, the percutaneous abutment, the threaded fastener, and the bone fixture are coaxial.

3. The apparatus of claim 1, wherein the active implant is coupled directly to the percutaneous abutment.

4. The apparatus of claim 1, wherein the active implant comprises an actuator and one or more processors.

5. The apparatus of claim 1, wherein the active implant overlaps both the percutaneous abutment and the active implant.

6. The apparatus of claim 1, wherein the percutaneous abutment comprises one or more sound input devices or one or more antennas.

7. The apparatus of claim 1, wherein the percutaneous abutment comprises electrical contacts configured to establish an electrical connection between an external device and the active implant when the external device is coupled to the percutaneous abutment.

8. The apparatus of claim 7, wherein the external device is configured to supply power and data to the active implant via the electrical connection.

9. The apparatus of claim 1, wherein the percutaneous abutment has a first section having a first degree of stiffness and a second section having a second degree of stiffness different from the first degree of stiffness.

10. The apparatus of claim 1, wherein the percutaneous abutment comprises a percutaneous abutment thread configured to mate with a bone fixture thread to couple the percutaneous abutment to the bone fixture.

11. A system comprising:
a percutaneous abutment comprising at least one supracutaneous electrical contact and at least one subcutaneous electrical contact;
an active implant comprising an implant power source and an active implant electrical contact configured to electrically couple to the at least one subcutaneous electrical contact; and
an external device comprising at least one external device electrical contact configured to electrically couple to the at least one supracutaneous electrical contact,
wherein the external device is configured to charge the implant power source via the at least one supracutaneous electrical contact.

12. The system of claim 11, wherein the active implant and the external device are configured to be laterally offset from each other along a recipient's skull when the active implant is implanted in the recipient and the external device is coupled to the percutaneous abutment.

13. The system of claim 11, wherein the external device is configured to supply power and data to the active implant by being electrically coupled to the at least one supracutaneous electrical contact.

14. The system of claim 11, further comprising:
a vibration damper configured to resist transmission of vibrations between the active implant and the external device.

15. The system of claim 11, wherein the percutaneous abutment comprises a first bend and a second bend configured to laterally offset the at least one supracutaneous electrical contact and the at least one subcutaneous electrical contact from each other.

16. An apparatus comprising:
a bone fixture configured to anchor to bone of a recipient;
a percutaneous abutment coupled to the bone fixture; and
an active implant disposed coaxially with the bone fixture and the percutaneous abutment,
wherein the active implant comprises a vibratory actuator.

17. The apparatus of claim 16, further comprising:
an external device comprising a microphone, a power source, and a sound processor;
wherein the external device is mechanically coupled to the percutaneous abutment;
wherein the external device is electrically coupled to the active implant via the percutaneous abutment; and
wherein the sound processor is configured to cause the vibratory actuator to actuate.

18. The apparatus of claim 16, further comprising:
an external device comprising an external device power source,
wherein the external device is configured to mechanically couple to the percutaneous abutment;
wherein the external device is configured to electrically couple to the active implant via the percutaneous abutment to charge an implanted power source of the active implant from the external device power source; and
wherein the percutaneous abutment comprises a supracutaneous microphone configured to supply data to the active implant.

19. The apparatus of claim 16,
a vibration damper configured to resist transmission of vibrations from the active implant along the percutaneous abutment.

20. The apparatus of claim 16,
wherein the apparatus further comprises an external device having an ear-hook and a cable extending from the external device, the cable (404) being electrically coupled to the active implant via the percutaneous abutment.
